(19) United States
(12) Patent Application Publication
(10) Pub. No.: US 2023/0023659 A1
(43) Pub. Date: Jan. 26, 2023

(54) CATALYST COMPONENT FOR ISOCYANATE MODIFICATION

(71) Applicant: Covestro Intellectual Property GmbH & Co. KG, Leverkusen (DE)

(72) Inventors: Frank RICHTER, Leverkusen (DE); Yoan PREUX, Livry-Gargan (FR)

(21) Appl. No.: 17/782,223

(22) PCT Filed: Dec. 15, 2020
(86) PCT No.: PCT/EP2020/086087

§ 371 Date: Jun. 3, 2022

(30) Foreign Application Priority Data
Dec. 19, 2019 (EP) 19217953.9

(51) Int. Cl. C08G 18/20; C08G 18/08

(52) U.S. Cl. CPC C08G 18/2018; C08G 18/14; C08G 2101/00

(57) ABSTRACT

The invention relates to a catalyst component for isocyanate modification, comprising at least one cyclic ammonium salt having a cation of the formula I wherein Y is a linear or branched C₂-C₂₀ segment which is substituted by a hydroxyl group in the 2 position to the charge-bearing nitrogen atom and optionally bears further substituents and is optionally interrupted by heteroatoms from the group of oxygen, sulfur, nitrogen and aromatic rings and optionally has further rings, and the N-bonded substituents R¹ and R² are either independently identical or different, substituted or unsubstituted, optionally branched, aliphatic C₁-C₂₀ radicals, aromatic C₆-C₂₀ radical or araliphatic C₇-C₂₀ radicals or the N-bonded substituents R¹ and R² form a ring segment X with one another for which the same or different definition given above for Y is applicable, with the proviso that X has a hydroxyl group as substituent in the 2 position to the charge-bearing nitrogen atom or does not have a hydroxyl group as substituent in the 2 position to the charge-bearing nitrogen atom.

The invention relates to a catalyst component for isocyanate modification, and to a process for modifying isocyanates in the presence of this catalyst component. The invention also relates to modified isocyanates themselves and to the use thereof for the production of polyurethane bodies or coatings, and also to the polyurethane bodies or coatings themselves. The invention further relates to one- or two-component systems comprising the modified isocyanates.

The oligomerization or polymerization of isocyanates, especially to form higher molecular weight oligomer mixtures having uretdione (“dimer”), isocyanurate (“trimer”) and/or iminooxadiazinedione structures (“asymmetric trimer”) in the molecular skeleton, has long been known. As can be seen above, the oligomerization and polymerization of isocyanates are based in principle on the same chemical reactions. The reaction of a relatively small number of isocyanates with one another is referred to as oligomerization. The reaction of a relatively large number of isocyanates is referred to as polymerization. In the context of the present invention, the oligomerization or polymerization of isocyanates described above is referred to collectively as isocyanate modification or modification of isocyanates.

The modified polyisocyanates comprising free NCO groups, which optionally may also have been temporarily deactivated with blocking agents, are exceptionally high-quality starting materials for the preparation of a multiplicity of polyurethane plastics and coating compositions.

A series of industrial methods for isocyanate modification have been established in which the isocyanate to be modified, usually a diisocyanate, is generally reacted by addition of catalysts and these are then rendered inactive (deactivated) by suitable measures, when the desired degree of conversion of the isocyanate to be modified has been reached, and the polyisocyanate obtained is generally separated from the unreacted monomer. A summary of these methods from the prior art can be found in H. J. Laas et al., J. Prakt. Chem. 1994, 336, 185 ff.

Compounds of ionic composition have proven to be effective as modification catalysts since they may be used in very low amounts, relative to the monomer to be converted, and lead extremely rapidly to the desired result, the cations being important in particular from the aspect of solubility of the respective salt in the isocyanate medium.

EP 1 170 283 A2 discloses a process for preparing polyisocyanurates, in which unstable catalysts are used in the isocyanate medium in order to avoid stopping the catalysts with inhibitors.

WO 2015/124504 A1 and WO 2017/029266 A1 describe very stable ammonium salts in which the charge-bearing nitrogen atom forms part of a ring system. However, these compounds have the disadvantage that they cannot be incorporated into isocyanate-functional compounds and thus remain as migration-capable impurities in the final process product, for example a polyurethane plastic or a polyurethane coating, and later give rise to undesirable effects such as fogging, etc.

The invention was therefore based on the object of providing a catalyst component for isocyanate modification which can be easily prepared from inexpensive reactants and which has a high catalytic activity and selectivity with simultaneously good catalyst stability and a low tendency towards the formation of disruptive byproducts in processes for isocyanate modification.

This object was achieved according to the invention by a catalyst component for isocyanate modification, comprising at least one cyclic ammonium salt with a cation of formula I,

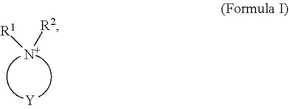

where
Y is a linear or branched C₂-C₂₀ segment which is substituted by a hydroxyl group in the 2 position to the charge-bearing nitrogen atom, optionally bears further substituents, is optionally interrupted by heteroatoms from the series oxygen, sulfur, nitrogen and also aromatic rings, and optionally includes further rings, and
the nitrogen substituents R¹ and R² either each independently are identical or different, substituted or unsubstituted, optionally branched, aliphatic C₁-C₂₀ radicals, aromatic C₆-C₂₀ radicals or araliphatic C₇-C₂₀ radicals, or
the nitrogen substituents R¹ and R² together form a ring segment X, for which the same or different definition given above for Y applies, with the proviso that X has a hydroxyl group in the 2 position to the charge-bearing nitrogen atom as substituent or does not have a hydroxyl group in the 2 position to the charge-bearing nitrogen atom as substituent.

The references to “comprising”, “containing”, etc., preferably denote “substantially consisting of” and very particularly preferably denote “consisting of”. The further embodiments identified in the claims and in the description can be combined arbitrarily, provided the context does not clearly indicate that the opposite is the case.

The charge-bearing nitrogen atom in formula I here is not part of the “further rings” that the C₂-C₂₀ segment of the cation of formula I may possibly include. In other words, the charge-bearing nitrogen atom in formula I is not a bridgehead atom of a tricycle.

In a first preferred embodiment, Y is a C₄-C₆ alkylene chain segment substituted by a hydroxyl group in the 2 position to the charge-bearing nitrogen atom and optionally bearing further substituents.

In a further preferred embodiment, R¹ and R² each independently are identical or different C₁-C₈ alkyl substituents or identical or different benzyl radicals optionally substituted on the aromatic ring, preferably are identical or different C₁-C₆ alkyl substituents and particularly preferably are identical or different C₁-C₆ alkyl substituents of linear structure.

In a further preferred embodiment, R¹ and R² together form a ring segment X, where X is a C₄-C₆ alkylene chain segment optionally bearing further substituents, with the proviso that X is substituted by a hydroxyl group in the 2 position to the charge-bearing nitrogen atom or is not substituted by a hydroxyl group in the 2 position to the charge-bearing nitrogen atom.

In a further preferred embodiment, the segment Y and/or the ring segment X are of linear structure.

In a further preferred embodiment, only Y has a hydroxyl group.

Cations of formula I, in which the nitrogen substituents R¹ and R² together form a ring system are spirocyclic compounds. These are obtainable in a simple manner by reaction of secondary cyclic amines with suitably substituted epoxy-functional haloalkanes and subsequent anion exchange.

X and Y in formula I may preferably each independently be optionally substituted alkylene groups, preference being given to C₄-C₆-alkylene chains, especially in both N-centered rings, and at least one of the two substituents X and/or Y bears an OH group. The C₄-C₆-alkylene chains are preferably of linear structure. These are obtainable in a simple manner, for example, by reaction of optionally C-substituted pyrrolidines, piperidines and azepanes (1H-hexahydroazepines) with 1,2-epoxy-4-halobutane, 1,2-epoxy-5-halopentane or 1,2-epoxy-6-halohexane and the C-substituted derivatives thereof, where halogen is Cl, Br and I, preferably Cl.

In addition, for example, by analogous reaction of optionally C-substituted oxazolidines, isoxazolidines, oxazinanes, morpholines and oxazepanes and the analogs of the aforementioned N—O heterocycles which contain S rather than O, and also imidazolidines, pyrazolidines, piperazines and structurally related compounds, with the abovementioned epoxy-functional haloalkanes, it is also possible to obtain representatives having C chains interrupted by heteroatoms in one of the segments X or Y of the general formula I. In the case of species containing 2 or more nitrogen atoms, it is additionally possible, by appropriate variation of the reaction conditions, also to produce salts having a doubly or multiply charged cation or, by prior suitable substitution of the nitrogen atom(s), to arrive at singly positively charged cations of formula I in which one or more exocyclic alkyl substituent(s) is/are present on the trivalent nitrogen atom(s) of the ring X or Y.

Of course, it is also possible through suitable choice of the alkylating agent to introduce a structural variation into the ring segment X or Y; examples include reactions of 1,2-epoxy-omega-haloalkyl ethers with the abovementioned secondary amines.

Compounds of formula I are obtainable in high yields by addition of secondary amines onto haloalkyloxiranes with simultaneously proceeding ring opening of the oxirane ring and quaternization of the nitrogen atom of the formerly secondary amine.

This synthesis route for obtaining cyclic, quaternary ammonium salts having a hydroxyl function in the 2 position to the charge-bearing nitrogen atom on the ring has not yet been described.

The present invention therefore further provides a process for preparing the catalyst component according to the invention, comprising the steps of

a) reacting an optionally cyclic secondary amine R¹, R²NH, in which R¹ and R² are the same substituents as in formula I,
b) optionally in the presence of a solvent,
c) with epoxy-functional haloalkanes to form the quaternary ammonium halides according to the invention, which can then optionally be converted in a step
d) by exchange of the halogen atom into ammonium compounds with any other anion.

DE 946 708 contains only a preliminary description for the formation of uncharged beta-oxypyrrolidines by reaction of ammonia or primary amines with beta-haloethylethylene oxides, preferably in the presence of bases. Those skilled in the art consequently have to assume that the base-induced elimination of HCl from the NH-functional ammonium chlorides possibly formed as intermediate is essential for the reaction to proceed.

This expectation is further supported by CN 105 151 266A, which describes the reaction of chloroalkyloxiranes with piperidine to give bis(piperidinyl)alkanols and contains no description of any cyclization with quaternization of the nitrogen atom.

JP 2012056897 describes the synthesis in poor yields of cyclic quaternary ammonium salts having a hydroxyl function on the ring by reaction of OH-functional secondary cyclic amines with alpha,omega-dihaloalkanes. Besides the poor yield, it is disadvantageous here that the OH-functional secondary cyclic amines required for the synthesis are significantly more difficult to obtain commercially than the OH-free base substances such as pyrrolidine or piperidine, whereas the haloalkyloxiranes to be used in the synthesis according to the invention are industrially readily available by simple oxidation (epoxidation) and easy to prepare haloolefins of suitable structure are obtainable without problems (see exemplary embodiment 1 of the present invention).

Preference is given to using reaction products of any secondary amines and 2-(2-haloethyl)oxiranes (II) or 2-(3-halopropyl)oxiranes (III)

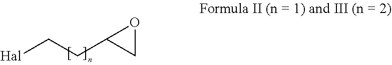

because the stability of 5- and 6-membered rings, which is generally known to organic chemists, leads to optimal results here. In addition, however, larger rings can also be synthesized, it possibly being necessary to operate in accordance with the Ruggli-Ziegler dilution principle. In formulae II and III, Hal is chlorine, bromine or iodine, preferably chlorine or bromine, particularly preferably chlorine.

Examples of reaction products with OH-functional 5-membered ring segment are obtainable from 1,2-epoxy-4-haloalkanes. Only the cations are given below (IUPAC names were generated by the BioVia/Draw, MDL.Draw.Editor 16.1.0.693 program): 1,1-dimethylpyrrolidin-1-ium-3-ol, 1,1-diethylpyrrolidin-1-ium-3-ol, 1,1-dibutylpyrrolidin-1-ium-3-ol, 5-azoniaspiro[4.4]nonan-3-ol, 5-azoniaspiro[4.5]decan-3-ol, 5-azoniaspiro[4.6]undecan-3-ol, 4,6,6-trimethylspiro[2-azoniabicyclo[2.2.2]octane-2,1′-azolidin-1-ium]-3′-ol, 8-oxa-5-azoniaspiro[4.5]decan-3-ol, 8-methyl-8-aza-5-azoniaspiro[4.5]decan-3-ol, 5,8-diazoniadispiro[4.2.48.25]tetradecane-3,11-diol.

Examples of reaction products with OH-functional 6-membered ring segment (from 1,2-epoxy-5-haloalkanes) include: 1,1-dimethylpiperidin-1-ium-3-ol, 1,1-diethylpiperidin-1-ium-3-ol, 1,1-dibutylpiperidin-1-ium-3-ol, 5-azoniaspiro[4.5]decan-9-ol, 6-azoniaspiro[5.5]undecan-4-ol, 6-azoniaspiro[5.6]dodecan-4-ol, 4,6,6-trimethylspiro[2-azoniabicyclo[2.2.2]octane-2,1′-azinan-1-ium]-3′-ol, 3-oxa-6-azoniaspiro[5.5]undecan-10-ol, 3-methyl-3-aza-6-azoniaspiro[5.5]undecan-10-ol and 6,9-diazoniadispiro[5.2.59.26]hexadecane-4,13-diol.

Anions used in the compounds of the formula I may in principle be any structure type known to be catalytically active with respect to isocyanates, preference being given to hydroxide, alkanoate, carboxylate, heterocycles having at least one negatively charged nitrogen atom in the ring, especially azolate, imidazolate, triazolate, tetrazolate, fluoride, hydrogendifluoride, higher polyfluorides or mixtures of these (adducts of more than one equivalent of HF onto compounds containing fluoride ions), the fluorides, hydrogendifluorides and higher polyfluorides leading in accordance with the invention to products having a high iminooxadiazinedione group content.

The catalyst component according to the invention is particularly suitable for isocyanate modification since in particular in the latter it has a high catalytic activity and selectivity with simultaneously good catalyst stability and a low tendency towards the formation of disruptive byproducts. The present invention therefore further provides a process for modifying isocyanates, in which at least one organic isocyanate having an NCO functionality of >1 is oligomerized in the presence of a catalyst component, characterized in that the catalyst component includes at least one cyclic ammonium salt with a cation of formula I,

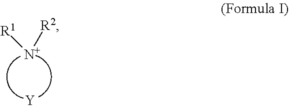

where
Y is a linear or branched C₂-C₂₀ segment which is substituted by a hydroxyl group in the 2 position to the charge-bearing nitrogen atom, optionally bears further substituents, is optionally interrupted by heteroatoms from the series oxygen, sulfur, nitrogen and also aromatic rings, and optionally includes further rings, and
the nitrogen substituents R¹ and R² either each independently are identical or different, substituted or unsubstituted, optionally branched, aliphatic C₁-C₂₀ radicals, aromatic C₆-C₂₀ radicals or araliphatic C₇-C₂₀ radicals, or
the nitrogen substituents R¹ and R² together form a ring segment X, for which the same or different definition given above for Y applies, with the proviso that X has a hydroxyl group in the 2 position to the charge-bearing nitrogen atom as substituent or does not have a hydroxyl group in the 2 position to the charge-bearing nitrogen atom as substituent. The embodiments of the cation of formula I that are mentioned above as preferred for the catalyst component are also preferred embodiments for the process according to the invention.

The catalysts according to the invention can be used individually or in any desired mixtures with one another. For instance, solutions of quaternary ammonium hydroxides in various alcohols, depending on the pKa of the base and of the alcohol used, are present partially or completely as ammonium salts with alkoxide anion. This equilibrium can be shifted entirely to the side of complete alkoxide formation by removing the water of reaction resulting from this reaction, it being possible in the individual case for this to also involve the ring-bonded OH function (betaine formation). Suitable methods for water removal are all methods known from the literature for this purpose, especially azeotropic distillation optionally with the aid of a suitable entrainer.

In the process according to the invention, it may further be provided that the oligomerization be conducted in the presence of a solvent.

For performance of the process according to the invention, it is possible in principle to use any known mono-, di- or polyisocyanates from the prior art, individually or in any desired mixtures with one another.

Examples include: pentamethylene diisocyanate (PDI), hexamethylene diisocyanate (HDI), 2-methylpentane 1,5-diisocyanate (MPDI), 2,4,4-trimethylhexane 1,6-diisocyanate and 2,2,4-trimethylhexane 1,6-diisocyanate (TMDI), 4-isocyanatomethyloctane 1,8-diisocyanate (nonane triisocyanate, NTI), 3(4)-isocyanatomethyl-1-methylcyclohexyl isocyanate (IMCI), isophorone diisocyanate (IPDI), 1,3- and 1,4-bis(isocyanatomethyl)benzene (XDI), 1,3- and 1,4-bis(isocyanatomethyl)cyclohexane (H₆XDI), norbornane diisocyanate (NBDI), tolylene 2,4- and 2,6-diisocyanate (TDI), bis(4-isocyanatophenyl)methane (4,4′MDI), 4-isocyanatophenyl-2-isocyanatophenylmethane (2,4′MDI) and polycyclic products obtainable by formaldehyde-aniline polycondensation and subsequent conversion of the resulting (poly)amines to the corresponding (poly)isocyanates (polymer MDI).

Preference is given to aromatic diisocyanates, i.e. diisocyanates in which both NCO groups are bonded to an sp²-hybridized carbon atom, or aliphatic diisocyanates, i.e. diisocyanates in which both NCO groups are bonded to an sp³-hybridized carbon atom.

Particular preference is given to PDI, HDI, MPDI, TMDI, NTI, IPDI, IMCI, XDI, H₆XDI, MDI, TDI or NBDI. Very particular preference is given to HDI, PDI or IPDI.

It is irrelevant by which methods the aforementioned isocyanates are generated, i.e. with or without use of phosgene.

The amount of the catalyst to be used in the process according to the invention is guided primarily by the organic isocyanate used and the desired reaction rate and is preferably between ≥0.001 and ≤5 mol %, preferably between ≥0.002 and ≤2 mol %, based on the sum total of the molar amounts of the isocyanate used and of the catalyst.

In the process according to the invention, the catalyst may be used undiluted or dissolved in solvents. Useful solvents are all compounds which do not react with the catalyst and are capable of dissolving it to a sufficient degree, for example optionally halogenated aliphatic or aromatic hydrocarbons, alcohols, ketones, esters and ethers. Preference is given to using alcohols.

The process according to the invention can be conducted within the temperature range from 0° C. to +250° C., preferably 20° C. to 200° C., particularly preferably 40° C. to 150° C., and can be interrupted at any degrees of conversion, preferably after 5% to 80%, particularly preferably 10% to 60%, of the isocyanate used has been converted.

Catalyst deactivation can be accomplished in principle by employing a whole series of previously described prior art methods, for example the addition of (sub-or super-)stoichiometric amounts of acids or acid derivatives (e.g. benzoyl chloride, acidic esters of phosphorus- or sulfur-containing acids, these acids themselves, etc., but not HF), adsorptive binding of the catalyst and subsequent removal by filtration, and other methods known to those skilled in the art.

In a further preferred embodiment, unconverted organic isocyanate is removed after deactivation of the catalyst system by any method of the prior art, for example by (thin film) distillation or extraction, and subsequently preferably reused.

In contrast to catalysis using open-chain, hydroxy-functional ammonium salts in which the charge-bearing nitrogen atom does not form part of a ring system (cf. e.g. EP 10589), surprisingly no notable reduction in catalytic activity is observed when using the catalysts according to the invention, even at relatively high reaction temperatures.

It is quite generally the case that the catalysts according to the invention, irrespective of the anion which is responsible for the catalytic activity and selectivity, are much more stable in the organic isocyanate to be converted than the prior art derivatives known from the literature.

According to a particular continuously operating embodiment of the process according to the invention, the oligomerization can be undertaken continuously, for example in a tubular reactor.

By the modification process according to the invention, a wide range of high-quality modified isocyanates, in the present case also referred to as polyisocyanates, which are therefore very valuable for the polyurethane sector, is very generally obtainable in a simple manner. The present invention further provides a modified isocyanate obtainable or prepared by the process according to the invention. The catalyst component according to the invention reacts via the OH group to form a covalent bond into the modified isocyanate. The present invention thus also provides a modified isocyanate, containing at least one structural element covalently bonded via the OH group of the cation of formula I from the catalyst component according to the invention as urethane and/or allophanate group. The embodiments of the cation of formula I that are mentioned above as preferred for the catalyst component are also preferred embodiments for the modified isocyanate according to the invention.

Depending on the starting (di)isocyanate used and the reaction conditions, the process according to the invention affords polyisocyanates of what is known as the isocyanate trimer type (i.e. containing isocyanurate and/or iminooxadiazinedione structures) having a low proportion of uretdione groups (“isocyanate dimers”). With rising reaction temperature, the proportion of the latter in the process products generally rises.

The products or product mixtures obtainable by the process according to the invention are consequently versatile starting materials for production of optionally foamed plastic(s) and of paints, coating compositions, adhesives and additives. Therefore, the present invention further provides for the use of the modified isocyanates according to the invention for the production of foamed or unfoamed plastics and paints, coating compositions, adhesives and additives. The catalysts according to the invention are also well suited for the production of optionally foamed polyurethane bodies with proportional formation of polyisocyanurate (what are known as PIR foams), since they are practically unable to migrate out of the finished products, which is important from the point of view of freedom from migration (leaching, fogging). Consequently, the present invention further provides polyurethane bodies obtainable or produced by reacting at least one monomeric diisocyanate and/or polyisocyanate with at least one polyol component in the presence of the catalyst component according to the invention. Where foamed polyurethane bodies are involved, preference is given to PIR foams.

The process products according to the invention can be used pure or in conjunction with other prior art isocyanate derivatives, such as polyisocyanates containing uretdione, biuret, allophanate, isocyanurate and/or urethane groups, the free NCO groups of which have optionally been deactivated with blocking agents.

The present invention further provides a one- or two-component system, containing a component A), comprising at least one modified isocyanate according to the invention, and a component B), comprising at least one NCO-reactive compound, and also a coating, obtainable or produced by curing a one- or two-component system according to the invention, optionally under the action of heat and/or in the presence of a catalyst, but also the substrates coated with at least one one- or two-component system according to the invention which has optionally been cured under the action of heat. Since the modified isocyanates according to the invention via the covalently bonded catalyst component according to the invention can also be found in the cured or foamed polyurethane bodies, the invention likewise provides a composite component comprising a material which is at least partly joined to a polyurethane body according to the invention or to a coating according to the invention.

In the present case, the term “modified isocyanate” has the meaning as defined at the outset and preferably represents a polyisocyanate having a statistical average of at least 1.5 NCO groups.

The comparative examples and examples which follow are intended to more particularly elucidate the invention without limiting it.

### EXAMPLES

All percentages, unless noted otherwise, are to be understood to mean percent by weight.

Mol % figures were determined by NMR spectroscopy and always relate, unless specified otherwise, to the sum total of the NCO conversion products. The measurements were effected on the Brucker DPX 400 or DRX 700 instruments on about 5% NMR) or about 50% (′³C NMR) samples in dry C₆D₆ at a frequency of 400 or 700 MHz (′H NMR) or 100 or 176 MHz CC NMR). The reference employed for the ppm scale was small amounts of tetramethylsilane in the solvent with 1H NMR chemical shift 0 ppm. Alternatively, the C₆D₅H present in the solvent was used as reference signal: NMR chemical shift 7.15 ppm; ¹³C NMR chemical shift 128.02 ppm. Data for the chemical shift of the compounds in question were taken from the literature (cf. D. Wendisch, H. Reiff and D. Dieterich, Die Angewandte Makromolekulare Chemie 141, 1986, 173-183 and literature cited therein and also EP 896 009 A1).

The dynamic viscosities were determined at 23° C. using a Haake VT 550 viscometer in accordance with DIN EN ISO 3219:1994-10. By measurements at different shear rates, it was ensured that the flow behavior of the polyisocyanate mixtures described according to the invention and also that of the comparative products corresponds to that of ideal Newtonian fluids. The indication of the shear rate can therefore be omitted.

The NCO content was determined by titration in accordance with DIN EN ISO 11909:2007-05.

The residual monomer contents were determined by gas chromatography in accordance with DIN EN ISO 10283:2007-11 using an internal standard.

All reactions were conducted under a nitrogen atmosphere unless stated otherwise.

The diisocyanates used are products of Covestro AG, D-51365 Leverkusen; all other commercially available chemicals were sourced from Aldrich, D-82018 Taufkirchen.

The reactants that were not commercially available were obtained by methods known from the literature.

Described by way of example here is the synthesis of catalysts 1 and 2 according to the following reaction sequence:

### Example 1: Preparation of Catalysts 1 and 2

10 g of 3-buten-1-ol (approx. 0.14 mol) were initially charged in a 100 ml two-neck flask at room temperature with stirring (magnetic stirrer), 7 drops (approx. 50 mg; approx 0.6 mmol) of pyridine were added thereto and the mixture was cooled to approx. 0° C. by immersion into an ice/water mixture. 17.8 g of thionyl chloride (approx. 0.15 mol) were then added dropwise slowly with stirring (magnetic stirrer), a light yellow coloration arising with brisk evolution of gas. The mixture was then stirred at 80° C. for a further 6 h, the now yellow-brown mixture was rapidly distilled at a slightly reduced pressure into a chilled receiver (−78° C., starting pressure 700 mbar, reduced in stages to 200 mbar) and the distillate obtained was then fractionated slowly at standard pressure over a 10 cm long Vigreux column. The 1-chloro-3-butene boiled at 75+/−1° C. 9.4 g (approx. 0.1 mol; approx. 75% yield, unoptimized) was obtained as colorless liquid.

This amount of 1-chloro-3-butene was then dissolved in approx. 150 ml of methylene chloride and a total of approx. 25 g of meta-chloroperoxybenzoic acid (77% purity according to manufacturer's data) was introduced in portions with vigorous mechanical stirring. After stirring for 24 hours at room temperature, the mixture was filtered, the filter residue was washed 3 times with approx. 100 ml of methylene chloride each time, the combined filtrates were rapidly distilled at slightly reduced pressure into a chilled receiver (−78° C., starting pressure 700 mbar, reduced in stages to 200 mbar) and the distillate obtained was then fractionated slowly over a 10 cm long Vigreux column first at standard pressure until virtually residue-free removal of the methylene chloride and then further at 50 mbar. The 1-chloro-3,4-butene oxide boiled at 70+/−2° C. 8.5 g (approx. 0.08 mol; approx. 80% yield, unoptimized) was obtained as colorless liquid.

This amount of 1-chloro-3,4-butene oxide was added dropwise with stirring (magnetic stirrer) to a refluxing mixture of 6.8 g of piperidine (approx. 0.08 mol) and approx. 150 g of water, and the mixture was stirred at 120° C. bath temperature for a further half an hour, freed from all volatile constituents under reduced pressure, and the remaining residue was divided without further purification (yield almost quantitative) and converted a) into the hydroxide by adding an aliquot of methanolic KOH solution and b) into the fluoride by adding a 100% excess of methanolic KF solution.

The solution obtained under a) was filtered, the precipitate was washed multiple times with 2-ethylhexanol (in the following text: 2-EH) and after concentration (freedom from MeOH checked by means of ¹H NMR) was adjusted to the desired content of catalyst 1 (see table 1).

The solution obtained under b) was filtered, the precipitate was washed multiple times with 2-EH and, after concentration (freedom from MeOH checked by means of ¹H NMR) adjusted to the desired content of catalyst 2 with 1.2 equivalents of a 10% solution of anhydrous HF in 2-EH and subsequent dilution with further 2-EH (see table 1).

Entirely analogously and with comparable yields, it is possible to synthesise further species starting from 4-penten-1-ol on the one hand and other secondary amines such as pyrrolidine or dimethylamine on the other (the aqueous solution was used and the operation was performed under slight positive pressure).

Further catalysts were obtained by analogous processes from the corresponding quaternary ammonium chlorides, which for their part had been prepared previously from the respective secondary, optionally cyclic, amine and the corresponding chloroalkyloxirane. Higher oligofluorides were obtained by adding appropriate excess HF to the (poly)fluoride solutions obtained in analogy to catalyst 2. Anions other than hydroxide or (poly)fluoride were obtained by salt metathesis with, for example, potassium acetate or potassium pivalate.

The optimal catalyst concentration for the isocyanate trimerization was determined in exploratory preliminary experiments at 60° C. with HDI (cf. example 2) and the concentration of the catalyst solution was adjusted by diluting with 2-EH such that only negligibly slight gel particle formation, if any, was observed when the catalyst solution was added to the HDI. An overview of the catalysts used can be found in table 1.

TABLE 1
Overview of the catalysts prepared (the catalyst concentration relates
to the active compound (cation and anion))

| Catalyst | Cation | Anion | Concentration [%] |
|---|---|---|---|
| 1 5-Azoniaspiro[4.5]decan-3- ol hydroxide | 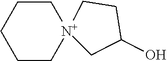 [SMILES: OC1CC[N+]2(CCCCC2)C1] | OH⁻ | 2 |
| 2 5-Azoniaspiro[4.5]decan-3- ol fluoride * 1.2 HF | 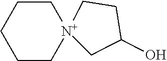 [SMILES: OC1CC[N+]2(CCCCC2)C1] | [F*1.2 HF]⁻ | 25 |
| 3 6-Azoniaspiro[5.5]undecan- 4-ol | 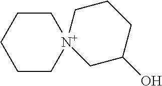 [SMILES: OC1CCC[N+]2(CCCCC2)C1] | (CH₃)₃CC(O)O⁻ | 10 |
| 4 6-Azoniaspor[5.5]undecan- 4-ol hydrogendifluoride | 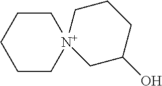 [SMILES: OC1CCC[N+]2(CCCCC2)C1] | [HF₂]⁻ | 30 |
| 5 1,1-Dimethylpyrrolidin-1- ium-3-ol hydroxide | 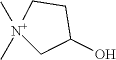 | OH⁻ | 1 |
| 6 1,1-Dimethylpyrrolidin-1- ium-3-ol hydrogendifluoride | 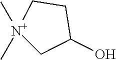 | [HF₂]⁻ | 10 |
| 7 1,1-Dimethylpiperidin-1- ium-3-ol pivalate | 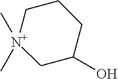 | CH₃C(O)O⁻ | 10 |
| 8 5-Azoniaspiro[4.4]nonan- 3-ol hydroxide | 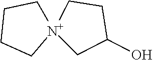 | OH⁻ | 1 |
| 9 5-Azoniaspiro[4.4]nonan- 3-ol hydrogendifluoride | 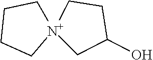 | [HF₂]⁻ | 15 |

### Examples 2 to 9—Isocyanate Modifications According to the Invention

A jacketed flange vessel heated to the starting temperature desired in each case by means of an external circuit, having a stirrer, reflux condenser connected to an inert gas system (nitrogen/vacuum) and thermometer, was initially charged with 1000 g of HDI which was freed of dissolved gases by stirring under reduced pressure (<1 mbar) for one hour. After venting with nitrogen, the type and amount of catalyst specified in table 2 was metered in, optionally in portions, in such a way that the maximum temperature specified in table 2 was not exceeded. After approx. 1 mol of NCO groups had been converted, as indicated by attainment of an NCO content of around 45.8%, the catalyst was deactivated by addition of an amount of the stopper specified in table 2 that was equivalent to the catalyst, and the mixture was stirred at reaction temperature for a further 30 min and subsequently worked up.

The workup was effected by vacuum distillation in a thin film evaporator of the short-path evaporator (SPE) type with an upstream preliminary evaporator (PE) (distillation data: pressure: 0.08+/−0.04 mbar, PE temperature: 120° C., ME temp.: 140° C.), with separation of unconverted monomer as distillate and the low-monomer polyisocyanate resin as bottom product (starting run). The polyisocyanate resin was separated and the distillate was collected in a second flange stirring apparatus of identical construction to the first, and made up to the starting amount (1000 g) with freshly degassed HDI. Thereafter, the mixture was treated again with catalyst and the procedure as described at the outset was followed. This procedure was repeated several times, optionally with variation of the reaction temperature (experiments A, B, C, etc.). The results can be found in table 2.

Finally, the distillate composition was ascertained by gas chromatography. In no case could decomposition products of the catalyst cation be detected (detection limit of approx. 20 ppm).

TABLE 2
Isocyanate modifications conducted

| | | | | Reaction | | |
|---|---|---|---|---|---|---|
| | | Catalyst | Amount of | temperature | | |
| Example | | (see table 1) | catalyst [g] | from-to [° C.] | | Stopper* |
| 1 | A | 1 | 4.3 | 60 | 62 | 1 |
| 1 | B | 1 | 5.7 | 60 | 71 | 1 |
| 1 | C | 1 | 6.2 | 80 | 90 | 1 |
| 1 | D | 1 | 5.9 | 80 | 85 | 1 |
| 1 | E | 1 | 6.5 | 100 | 120 | 1 |
| 1 | F | 1 | 6.8 | 100 | 140 | 1 |
| 2 | A | 2 | 0.85 | 60 | 64 | 3 |
| 2 | B | 2 | 0.81 | 60 | 63 | 3 |
| 2 | C | 2 | 0.75 | 80 | 88 | 3 |
| 2 | D | 2 | 0.61 | 80 | 83 | 3 |
| 2 | E | 2 | 0.62 | 100 | 125 | 3 |
| 2 | F | 2 | 0.61 | 100 | 104 | 3 |
| 3 | A | 3 | 1.42 | 60 | 60 | 1 |
| 3 | B | 3 | 1.82 | 60 | 64 | 1 |
| 3 | C | 3 | 2.11 | 80 | 85 | 1 |
| 3 | D | 3 | 2.00 | 80 | 84 | 1 |
| 3 | E | 3 | 2.10 | 100 | 137 | 1 |
| 3 | F | 3 | 2.22 | 100 | 101 | 1 |
| 4 | A | 4 | 0.91 | 60 | 61 | 2 |
| 4 | B | 4 | 0.95 | 60 | 62 | 2 |
| 4 | C | 4 | 0.69 | 60 | 61 | 2 |
| 4 | D | 4 | 0.67 | 60 | 61 | 2 |
| 4 | E | 4 | 0.60 | 60 | 61 | 2 |
| 4 | F | 4 | 0.58 | 60 | 61 | 2 |
| 5 | A | 5 | 8.5 | 60 | 77 | 1 |
| 5 | B | 5 | 9.2 | 60 | 62 | 1 |
| 5 | C | 5 | 9.8 | 80 | 88 | 1 |
| 5 | D | 5 | 9.5 | 80 | 92 | 1 |
| 5 | E | 5 | 10.2 | 100 | 124 | 1 |
| 5 | F | 5 | 10.9 | 100 | 103 | 1 |
| 6 | A | 6 | 1.71 | 60 | 62 | 2 |
| 6 | B | 6 | 1.42 | 60 | 61 | 2 |
| 6 | C | 6 | 1.25 | 80 | 85 | 2 |
| 6 | D | 6 | 1.02 | 80 | 83 | 2 |
| 6 | E | 6 | 0.95 | 100 | 102 | 2 |
| 6 | F | 6 | 0.98 | 100 | 105 | 2 |
| 7 | A | 7 | 1.32 | 60 | 62 | 1 |
| 7 | B | 7 | 1.65 | 60 | 61 | 1 |
| 7 | C | 7 | 1.98 | 80 | 83 | 1 |
| 7 | D | 7 | 1.98 | 80 | 82 | 1 |
| 7 | E | 7 | 1.89 | 100 | 104 | 1 |
| 7 | F | 7 | 2.01 | 100 | 104 | 1 |
| 8 | A | 8 | 8.7 | 60 | 62 | 1 |
| 8 | B | 8 | 9.4 | 60 | 61 | 1 |
| 8 | C | 8 | 10.1 | 80 | 83 | 1 |
| 8 | D | 8 | 10.2 | 80 | 82 | 1 |
| 8 | E | 8 | 11.0 | 100 | 104 | 1 |
| 8 | F | 8 | 10.8 | 100 | 104 | 1 |
| 9 | A | 9 | 1.12 | 60 | 62 | 3 |
| 9 | B | 9 | 1.20 | 60 | 61 | 3 |
| 9 | C | 9 | 1.02 | 80 | 83 | 3 |
| 9 | D | 9 | 0.98 | 80 | 82 | 3 |
| 9 | E | 9 | 0.82 | 100 | 104 | 3 |
| 9 | F | 9 | 0.84 | 100 | 104 | 3 |

*Stopper:
1: dibutyl phosphate,
2: toluenesulfonic acid, 40% in 2-PrOH,
3: dodecylbenzenesulfonic acid, 70% in 2-PrOH

The resins obtained were, without exception, light-colored clear viscous liquids with no perceptible amine odor and devoid of extractable catalyst byproducts or conversion products. In the case of use of the fluorine-containing catalysts, the result was mixtures of isocyanurate and iminooxadiazinedione along with a little uretdione. The catalysts with oxygen-containing anions afford products of the isocyanurate type, where, as is the case with the use of the fluorine-containing catalysts, the alcohol (2-EH) used as catalyst solvent is completely converted to the allophanate.

The recovered HDI is free of impurities that result from the decomposition of the catalyst cation and can be reused without problems in the same or in different processes.

1: A catalyst component for isocyanate modification, comprising at least one cyclic ammonium salt with a cation of formula I, 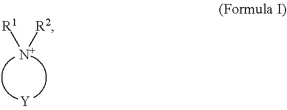
where Y is a linear or branched C₂-C₂₀ segment which is substituted by a hydroxyl group in the 2 position to the charge-bearing nitrogen atom, optionally bears further substituents, is optionally interrupted by heteroatoms from the series oxygen, sulfur, nitrogen and also aromatic rings, and optionally includes further rings, and
the nitrogen substituents R¹ and R² either each independently are identical or different, substituted or unsubstituted, optionally branched, aliphatic C₁-C₂₀ radicals, aromatic C₆-C₂₀ radicals or araliphatic C₇-C₂₀ radicals, or
the nitrogen substituents R¹ and R² together form a ring segment X, for which the same or different definition given above for Y applies, with the proviso that X has a hydroxyl group in the 2 position to the charge-bearing nitrogen atom as substituent or does not have a hydroxyl group in the 2 position to the charge-bearing nitrogen atom as substituent.

2: The catalyst component as claimed in claim 1, characterized in that Y is a C₄-C₆ alkylene chain segment substituted by a hydroxyl group in the 2 position to the charge-bearing nitrogen atom and optionally bearing further substituents.

3: The catalyst component as claimed in claim 1, characterized in that R¹ and R² each independently are identical or different C₁-C₈ alkyl substituents or identical or different benzyl radicals optionally substituted on the aromatic ring.

4: The catalyst component as claimed in claim 1, characterized in that R¹ and R² together form a ring segment X, where X is a C₄-C₆ alkylene chain segment optionally bearing further substituents, with the proviso that X is substituted by a hydroxyl group in the 2 position to the charge-bearing nitrogen atom or is not substituted by a hydroxyl group in the 2 position to the charge-bearing nitrogen atom.

5: The catalyst component as claimed in claim 1, characterized in that the segment Y and/or the ring segment X are of linear structure.

6: The catalyst component as claimed in claim 1, characterized in that only Y has a hydroxyl group.

7: A process for modifying isocyanates, in which at least one organic isocyanate having an NCO functionality of >1 is oligomerized and/or polymerized in the presence of a catalyst component as claimed in claim 1.

8: The process as claimed in claim 7, characterized in that the organic isocyanate is an organic diisocyanate and is selected from the group consisting of PDI, HDI, MPDI, TMDI, NTI, IPDI, IMCI, XDI, H6XDI, MDI, TDI, and NBDI.

9: A modified isocyanate, containing at least one structural element covalently bonded via the OH group of the cation of formula I as urethane and/or allophanate group, wherein the cation of formula I has a structure as claimed in claim 1.

10: In a process for the production of foamed or unfoamed plastics and paints, coating compositions, adhesives or additives, the improvement comprising including at least one modified isocyanate as claimed in claim 9.

11: A one- or two-component system, containing a component A), comprising at least one modified isocyanate as claimed in claim 9, and a component B), comprising at least one compound reactive towards NCO groups.

12: A polyurethane body produced by reacting at least one of a monomeric diisocyanate and a polyisocyanate with at least one polyol component in the presence of the catalyst component as claimed in claim 1.

13: The polyurethane body as claimed in claim 12, characterized in that the polyurethane body is a foamed polyurethane body.

14: A coating produced by curing a one- or two-component system as claimed in claim 11, optionally under the action of heat and/or in the presence of a catalyst.

15: A composite component comprising a material which is at least partly joined to a polyurethane body as claimed in claim 12.
